(19) United States
(12) Patent Application Publication
(10) Pub. No.: US 2023/0085337 A1
(43) Pub. Date: Mar. 16, 2023

(54) BIOMARKER COMPUTING DEVICE AND BIOMARKER COMPUTING METHOD

(71) Applicant: PANASONIC INTELLECTUAL PROPERTY MANAGEMENT CO., LTD., Osaka (JP)

(72) Inventors: Tadanori TEZUKA, Fukuoka (JP); Tsuyoshi NAKAMURA, Fukuoka (JP); Yukihiro MORITA, Osaka (JP)

(73) Assignee: PANASONIC INTELLECTUAL PROPERTY MANAGEMENT CO., LTD., Osaka (JP)

(21) Appl. No.: 17/801,745

(22) PCT Filed: Feb. 17, 2021
(86) PCT No.: PCT/JP2021/006015

§ 371 Date: Aug. 23, 2022

(30) Foreign Application Priority Data
Feb. 25, 2020 (JP) 2020-029882

(51) Int. Cl. G16H 50/30

(52) U.S. Cl. CPC G16H 50/30

(57) ABSTRACT

A biomarker computing device includes an input unit configured to input biometric data of a subject measured by a biosensor, a biomarker estimation unit configured to estimate, using the input biometric data in a first period, a biomarker indicating a physical and mental state of the subject, a computation unit configured to collect, in a second period longer than the first period, a plurality of biomarker estimation results estimated in each first period, and calculate, based on appearance frequencies of the physical and mental states indicated respectively by the plurality of biomarker estimation results, a proportion of each of the physical and mental states, a graph creation unit configured to create a graph showing the proportion of each of the physical and mental states, and a display control unit configured to display the created graph on a display device.

### TECHNICAL FIELD

The present disclosure relates to a biomarker computing device and biomarker computing method for computing a biomarker based on sensing data related to biological information of a person.

### BACKGROUND ART

Patent Literature 1 discloses an analysis support apparatus that creates time-series graph data based on a marker of an emotional state of a subject calculated for each predetermined sampling interval, associates viewpoint coordinate data with a scene image frame by frame, synchronizes the time series based on a time difference, and displays the created graph of the marker of the emotional state and the scene image associated with the viewpoint coordinates on a display unit on the same screen. The analysis support apparatus simultaneously displays markers of one or more emotional states of the subject on a radar chart. Therefore, a correlation between markers, quantitative and temporal relationships of undulations, and the like can be visually confirmed, and can be used for an analysis.

### CITATION LIST

### Patent Literature

Patent Literature 1: JP-A-2015-188649

### SUMMARY OF INVENTION

### Technical Problem

In Patent Literature 1, the markers of the emotional states are displayed on a graph such as a radar chart by accumulating biomarker measurement data of a certain period, for example. However, it may be difficult to summarize the biomarkers of the subject in a unit period only by displaying the biomarker measurement data accumulated for a certain period on the graph. For example, there is a problem in intuitively and visually showing how a change of the biomarker of a certain subject in one day of a certain day is balanced, or showing a transition about how the biomarker of a certain subject changes through one month.

The present disclosure has been made in view of the above-described circumstances, and an object thereof is to provide a biomarker computing device and biomarker computing method for outputting a summary result of a biomarker of a subject in a unit period and efficiently supporting an analysis of the biomarker of the subject.

### Solution to Problem

The present disclosure provides a biomarker computing device including: an input unit configured to input biometric data of a subject measured by a biosensor; a biomarker estimation unit configured to estimate, by using the input biometric data in a first period, a biomarker indicating a physical and mental state of the subject; a computation unit configured to collect, in a second period longer than the first period, a plurality of biomarker estimation results estimated in each first period, and calculate, based on appearance frequencies of the physical and mental states indicated respectively by the plurality of biomarker estimation results, a proportion of each of the physical and mental states; a graph creation unit configured to create a graph showing the proportion of each of the physical and mental states; and a display control unit configured to display the created graph on a display device.

Further, the present disclosure provides a biomarker computing method to be performed by a biomarker computing device, and the biomarker computing method includes: a step of inputting biometric data of a subject measured by a biosensor; a step of estimating, by using the input biometric data in a first period, a biomarker indicating a physical and mental state of the subject; a step of collecting, in a second period longer than the first period, a plurality of biomarker estimation results estimated in each first period, and calculating, based on appearance frequencies of the physical and mental states indicated respectively by the plurality of biomarker estimation results, a proportion of each of the physical and mental states; a step of creating a graph showing the proportion of each of the physical and mental states; and a step of displaying the created graph on a display device.

### Advantageous Effects of Invention

According to the present disclosure, a summary result of a biomarker of a subject in a unit period can be output, and an analysis of the biomarker of the subject can be efficiently supported.

### BRIEF DESCRIPTION OF DRAWINGS

FIG. 1 is a block diagram showing a configuration example of a biomarker monitoring system according to a first embodiment.

FIG. 2 is a diagram showing an example of RRI data obtained for a certain subject.

FIG. 3 is a diagram showing examples of biomarker estimation results and proportion calculation results based on sensing result data accumulated in a second period.

FIG. 4 is a diagram showing an example of a Lorenz plot of the RRI data.

FIG. 5 is a diagram showing a calculation example of an average and a deviation based on the Lorenz plot of the RRI data.

FIG. 6 is a diagram showing an example of a time-series plot of a pair including an average and a deviation calculated for each second period.

FIG. 7 is a diagram showing a first calculation example of obtaining a change direction (movement vector) for each first period based on the time-series plot of FIG. 6.

FIG. 8 is a diagram showing a second calculation example of obtaining the change direction (movement vector) for each first period based on the time-series plot of FIG. 6.

FIG. 9 is a diagram showing an example of an eight-classification map in which the movement vectors of FIG. 7 or FIG. 8 are aligned with a center point O.

FIG. 10A is a diagram of a radar chart showing an example of appearance frequencies of a plurality of biomarkers of a subject in a unit period.

FIG. 10B is a diagram of a radar chart showing an example of appearance frequencies of a plurality of biomarkers of a subject in a unit period.

FIG. 10C is a diagram of a pie chart showing an example of appearance frequencies of biomarkers of a subject in a unit period.

FIG. 10D is a diagram of a bar graph showing an example of appearance frequencies of biomarkers of a subject in a unit period.

FIG. 10E is a diagram of a line graph showing an example of appearance frequencies of biomarkers of a subject in a unit period.

FIG. 11 is a flowchart showing an example of an overall operation procedure of a biomarker computing device according to the first embodiment.

FIG. 12 is a flowchart showing a first example of a cumulative calculation process of FIG. 11.

FIG. 13 is a flowchart showing a second example of the cumulative calculation process of FIG. 11.

### DESCRIPTION OF EMBODIMENTS

Hereinafter, embodiments specifically disclosing a biomarker computing device and a biomarker computing method according to the present disclosure will be described in detail with reference to the drawings as appropriate. An unnecessarily detailed description may be omitted. For example, a detailed description of a well-known matter or a repeated description of substantially the same configuration may be omitted. This is to avoid unnecessary redundancy in the following description and to facilitate understanding of those skilled in the art. It should be noted that the accompanying drawings and the following description are provided for a thorough understanding of the present disclosure by those skilled in the art, and are not intended to limit the subject matter recited in the claims.

In the following embodiment, a use case will be described as an example in which an employee who works on a desk in a certain office is set as a subject, daily physical and mental states of the employee are monitored (calculated) using biometric data thereof, and proportions of the states of the employee calculated during a certain period are indicated by a graph. However, the subject is not limited to an employee in an office, and may be, for example, a store clerk at a checkout counter of a store.

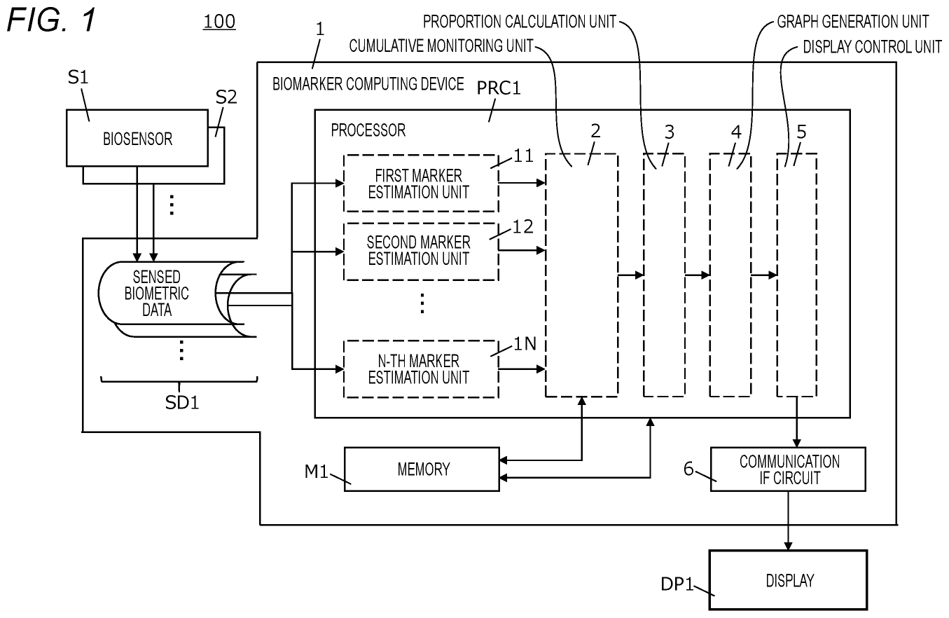

FIG. 1 is a block diagram showing a configuration example of a biomarker monitoring system 100 according to a first embodiment. The biomarker monitoring system 100 includes biosensor S1, S2, . . . , a biomarker computing device 1, and a display DP1. The biosensors S1, S2, . . . are connected to the biomarker computing device 1 by a wire such as a wired LAN (Local Area Network) and further the biomarker computing device 1 is connected to the display DP1 by a wire such as a wired LAN (Local Area Network). The biosensors S1, S2, . . . may be connected to the biomarker computing device 1 wirelessly instead of by wire, and further the biomarker computing device 1 may be connected to the display DP1 wirelessly instead of by wire.

The biosensors S1, S2, . . . are sensors (measuring devices) that measure and acquire biometric data of the subject, and are, for example, a heart rate meter, an electroencephalograph, a skin potential meter, or an electroencephalogram and Korotkoff sound measuring device. The heart rate meter measures variation (RRI) data of heartbeat sounds of the subject as sensed biometric data SD1 by winding an electrode around a wrist or a neck of the subject. The electroencephalograph measures brain wave data of the subject as the sensed biometric data SD1 based on signals obtained from an electrode that is in contact with a head of the subject (specifically, a part that gives a reaction related to emotions or feelings, such as a left front frontal lobe). The skin potential meter measures skin potential data on skin at such as a forearm of the subject as the sensed biometric data SD1. Similarly to a blood pressure measuring device, the electroencephalogram and Korotkoff sound measuring device measures data of brain waves and Korotkoff sounds as the sensed biometric data SD1 with one cuff (arm band) wound around an upper arm of the subject near an elbow joint. The biosensors S1, S2, . . . send the RRI data, the brain wave data, the skin potential data, the data of brain waves and Korotkoff sounds of the subject to the biomarker computing device 1 as sensed biometric data. The biosensors S1, S2, . . . may include a monitoring camera capable of capturing a color image of the subject. As disclosed in, for example, JP-B-6358506 or JP-B-6323809, the monitoring camera can measure the variation (RRI) data of the heartbeat sounds of the subject in a non-contact manner based on the captured color image of the subject, and transmits the RRI data to the biomarker computing device 1 as the sensed biometric data.

The biomarker computing device 1 receives the sensed biometric data of the subject measured by the biosensors S1, S2, . . . , and estimates a biomarker indicating a physical and mental state of the subject using the received sensed biometric data in a first period (refer to the following description). The biomarker computing device 1 collects, in a second period (refer to the following description) longer than the first period, a plurality of biomarker estimation results estimated in each first period, and calculates, based on appearance frequencies of the physical and mental states indicated respectively by the plurality of biomarker estimation results, a proportion of each of the physical and mental states. The biomarker computing device 1 creates a graph showing the proportion of each of the physical and mental states, and displays the graph on the display DP1. The biomarker computing device 1 includes a processor PRC1, a memory M1, and a communication IF circuit 6. In FIG. 1, the biomarker computing device 1 is shown to be separated from the display DP1, but the biomarker computing device 1 may have a configuration in which the display DP1 is incorporated.

The biomarker computing device 1 receives and stores the sensed biometric data SD1 of the subject measured by the biosensors S1, S2, . . . . In FIG. 1, the sensed biometric data SD1 is input to the processor PRC1 as an example of an input unit, but may be stored in the memory M1.

The processor PRC1 is implemented by using, for example, a central processing unit (CPU), a digital signal processor (DSP), or a field programmable gate array (FPGA). The processor PRC1 includes a first marker estimation unit 11, a second marker estimation unit 12, . . . an N-th marker estimation unit 1N, a cumulative monitoring unit 2, a proportion calculation unit 3, a graph creation unit 4, and a display control unit 5. The first marker estimation unit 11, the second marker estimation unit 12, . . . the N-th marker estimation unit 1N, the cumulative monitoring unit 2, the proportion calculation unit 3, the graph creation unit 4, and the display control unit 5 are functionally constructed in the processor PRC1 by the processor PRC1 reading and executing programs and data stored in a ROM (refer to the following description) of the memory M1. The first marker estimation unit 11, the second marker estimation unit 12, . . . and the N-th marker estimation unit 1N are configured as a total of N (N is an integer equal to or greater than 2) marker estimation units. When N=2, the second marker estimation unit 12 is the N-th marker estimation unit 1N.

Each of the first marker estimation unit 11, the second marker estimation unit 12, . . . , and the N-th marker estimation unit 1N as an example of a biomarker estimation unit receives the sensed biometric data SD1 of the same subject or different subjects, and estimates a biomarker indicating a physical and mental state (for example, “relaxation”, “tension”, or the like to be described later) of the subject using the sensed biometric data SD1. For example, the first marker estimation unit 11 receives the RRI data (refer to the above) at any time, the second marker estimation unit 12 receives the RRI data (refer to the above) and the brain wave data at any time, and similarly, the N-th marker estimation unit 1N receives the sensed biometric data SD1 of another combination different from a combination of the sensed biometric data SD1 received by each of the first marker estimation unit 11 to the (N−1)-th marker estimation unit (not shown) at any time. Biomarker estimation methods respectively performed by the first marker estimation unit 11, the second marker estimation unit 12, and the N-th marker estimation unit 1N are different.

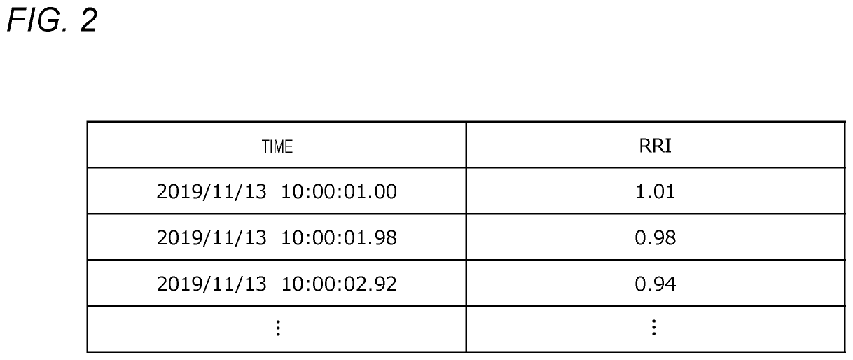

The first marker estimation unit 11 uses the RRI data (refer to FIG. 2) in a first period (for example, 5 minutes, 15 minutes, 30 minutes, or one hour) among the RRI data received at any time, to estimate a biomarker indicating a physical and mental state of the subject in the first period, and transmits the estimation result to the cumulative monitoring unit 2.

FIG. 2 is a diagram showing an example of the RRI data obtained for a certain subject. The RRI data in FIG. 2 includes, for example, data strings each including a date time when the heartbeat sound is measured and the RRI data. In FIG. 2, for example, the RRI of the subject at 10:00:01:00 on Nov. 13, 2019 is “1.01”, the RRI of the subject at 10:00:01:98 on Nov. 13, 2019 is “0.98”, and the RRI of the subject at 10:01:02:92 on Nov. 13, 2019 is “0.94”.

The physical and mental state (that is, the biomarker) is a physical or mental state of the subject, and corresponds to, for example, “relaxation”, “high performance”, “tension”, “concentration”, and “poor performance”, but is not limited thereto. The first period is set in advance by an operation of an administrator of the biomarker monitoring system 100, is not limited to the above-described time, and is a collection period of the sensed biometric data SD1 necessary for the first marker estimation unit 11 to estimate the biomarker of the subject. That is, the first period corresponds to a shortest estimation period of an estimation process performed by the first marker estimation unit 11. The biomarker estimation result is output in, for example, binary data (that is, data formats of “0” and “1”) for each biomarker. For example, when the biomarker estimated using the sensed biometric data SD1 in a certain first period indicates “relaxation”, the “relaxation” is “1”, and each of the “high performance”, the “tension”, the “concentration”, and the “poor performance” is “0”. Some patterns of heart rate variability may overlap, such as “tension” and “concentration”, and in such a case, when the biomarker estimation result is handled in the form of binary data of “0” and “1”, a result of “tension”=“1” and “concentration”=“1” is output. As to be described later, in a case of multiple values instead of binary data, a result of “tension”=“0.5”, “concentration”=“0.5”, “0.6”, or “0.4” may be output.

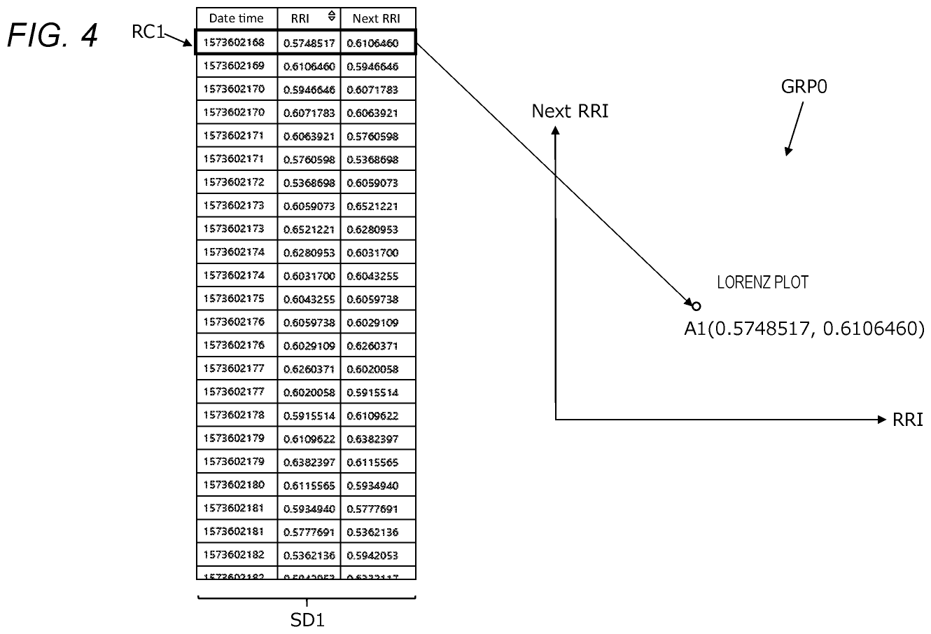

The “relaxation” is a state in which a heartbeat decreases and a fluctuation of the heartbeat is large within the first period, and specifically indicates a state in which the subject is relaxed. The “high performance” is a state in which the heartbeat rises and the fluctuation of the heartbeat is large within the first period, and specifically indicates a state in which the subject can show high performance on things to be done such as work. The “tension” is a state in which the heartbeat rises and the fluctuation of the heartbeat is small within the first period, and specifically indicates a state in which the subject is slightly tensed. The “concentration” is a state in which there is little vertical fluctuation of the heartbeat within the first period, and specifically indicates a state in which the subject is concentrated. The “poor performance” is a state in which the heartbeat decreases and the fluctuation of the heartbeat is small within the first period, and specifically indicates a state in which the subject is unmotivated or sleepy on things to be done such as work. A method of estimating the biomarker by the first marker estimation unit 11 will be described later in detail with reference to FIGS. 4 to 9.

When collecting the sensed biometric data SD1 of a combination different from that of the first marker estimation unit 11 in a first period, each of the second marker estimation unit 12 to the N-th marker estimation unit 1N estimates the biomarker indicating the physical and mental state of the subject by a method different from that of the first marker estimation unit 11, and transmits the estimation result for the first period to the cumulative monitoring unit 2. The estimation method performed by each of the second marker estimation unit 12 to the N-th marker estimation unit 1N may be the same as that performed by the first marker estimation unit 11, or may be different from that performed by the first marker estimation unit 11 (for example, a known method). For example, a stress marker such as LF/HF may be calculated using the RRI data in the first period, and a biomarker indicating the physical and mental state of the subject may be estimated.

The cumulative monitoring unit 2 as an example of a computation unit determines whether the biomarker estimation results of the subject estimated by the first marker estimation unit 11 to the N-th marker estimation unit 1N for each first period are accumulated and collected for a second period (for example, one hour, one day, one week, or one month). The second period is set in advance by an operation of the administrator of the biomarker monitoring system 100 so as to be longer than the first period, is not limited to the above-described time, and is an example of a unit period in which the estimation result is displayed in a graph created by the graph creation unit 4 to be described later. The cumulative monitoring unit 2 may temporarily store, into the memory M1, the biomarker estimation result of the subject estimated by each of the first marker estimation unit 11 to the N-th marker estimation unit 1N for each first period. For example, in a case where the first period is one hour and the second period is one day, the cumulative monitoring unit 2 determines whether the biomarker estimation results in a total of 24 hours (that is, one day), which are estimated for every one hour by each of the first marker estimation unit 11 to the N-th marker estimation unit 1N, are collected. When it is determined that the biomarker estimation results in one second period from each of the first marker estimation unit 11 to the N-th marker estimation unit 1N are collected, the cumulative monitoring unit 2 sends the biomarker estimation results in one second period from each of the first marker estimation unit 11 to the N-th marker estimation unit 1N to the proportion calculation unit 3.

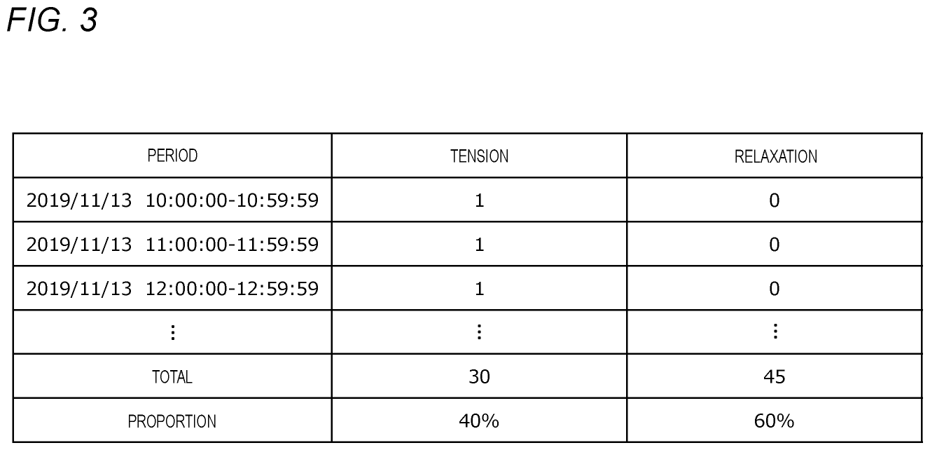

The proportion calculation unit 3 as an example of the computation unit counts an appearance frequency of each of a plurality of biomarkers in the second period by using the biomarker estimation results in one second period from each of the first marker estimation unit 11 to the N-th marker estimation unit 1N. The proportion calculation unit 3 calculates a proportion of each biomarker (that is, the physical and mental state) based on a count value of the appearance frequency of each of the plurality of biomarkers in the second period (refer to FIG. 3).

FIG. 3 is a diagram showing an example of biomarker estimation results and proportion calculation results based on the sensing result data accumulated in the second period. In the example of FIG. 3, the first period is one hour, and the second period is one day. At “10:00:00 to 10:59:59 on Nov. 13, 2019”, the biomarkers of the subject were “tension” of “1” and “relaxation” of “0”. At “11:00:00 to 11:59:59 on Nov. 13, 2019”, the biomarkers of the subject were “tension” of “1” and “relaxation” of “0”. Similarly, at “12:00:00 to 12:59:59 on Nov. 13, 2019”, the biomarkers of the subject were “tension” of “1” and “relaxation” of “0”.

The proportion calculation unit 3 calculates total appearance frequencies of the respective biomarkers “tension” and “relaxation” in the second period (that is, “Nov. 13, 2019” corresponding to “one day”) as “30” and “45”. In FIG. 3, only “tension” and “relaxation” are shown as the biomarkers for simplification of description, but total appearance frequencies of the respective remaining three biomarkers “high performance”, “concentration”, and “poor performance” may be included. The proportion calculation unit 3 calculates a proportion of each of the biomarkers “tension” and “relaxation” by using “30” and “45” which are respectively appearance frequencies of the biomarkers “tension” and “relaxation”. Specifically, the proportion calculation unit 3 calculates the proportion of the biomarker “tension” as 40% (=30/(30+45)), and calculates the proportion of the biomarker “relaxation” as 60% (=45/(30+45)). The proportion calculation unit 3 sends the calculation result of the proportion of each biomarker to the graph creation unit 4.

The biomarker estimation result acquired by the proportion calculation unit 3 from the first marker estimation unit 11 to the N-th marker estimation unit 1N via the cumulative monitoring unit 2 may be output in the form of binary data. However, when ranges of values of the biomarker estimation results acquired from the first marker estimation unit 11 to the N-th marker estimation unit 1N are the same range (for example, values of “0” to “1”), the biomarker estimation results do not have to be in the format of binary data. In this case, the proportion calculation unit 3 may convert the values of the estimation results from the first marker estimation unit 11 to the N-th marker estimation unit 1N into the format of binary data (that is, a value of “1” or a value of “0”) by a known method. Accordingly, data formats of the biomarker estimation results obtained from the first marker estimation unit 11 to the N-th marker estimation unit 1N are standardized, so that the efficiency of the calculation process at the time of the proportion calculation in the proportion calculation unit 3 is accurately improved.

In addition, when the ranges of the values of the biomarker estimation results acquired from the first marker estimation unit 11 to the N-th marker estimation unit 1N are the same range (for example, values of “0” to “1”), the proportion calculation unit 3 may accumulate the values of the biomarker estimation results acquired from the first marker estimation unit 11 to the N-th marker estimation unit 1N as they are even if the biomarker estimation results acquired from the first marker estimation unit 11 to the N-th marker estimation unit 1N do not have the format of binary data. Accordingly, the proportion calculation unit 3 can easily calculate the proportion by using the biomarker estimation results obtained from the first marker estimation unit 11 to the N-th marker estimation unit 1N as they are.

When the ranges of the values of the biomarker estimation results acquired from the first marker estimation unit 11 to the N-th marker estimation unit 1N are not the same range (for example, values of “0” to “1”), the proportion calculation unit 3 may normalize the biomarker estimation results acquired from the first marker estimation unit 11 to the N-th marker estimation unit 1N such that, for example, the biomarker estimation results have values in the same range described above. Thereafter, the proportion calculation unit 3 may calculate the proportion using the normalized values of the biomarker estimation results. Accordingly, the proportion calculation unit 3 can calculate the biomarker estimation result of the subject with high accuracy regardless of a type of the biomarker estimation method of each of the first marker estimation unit 11 to the N-th marker estimation unit 1N.

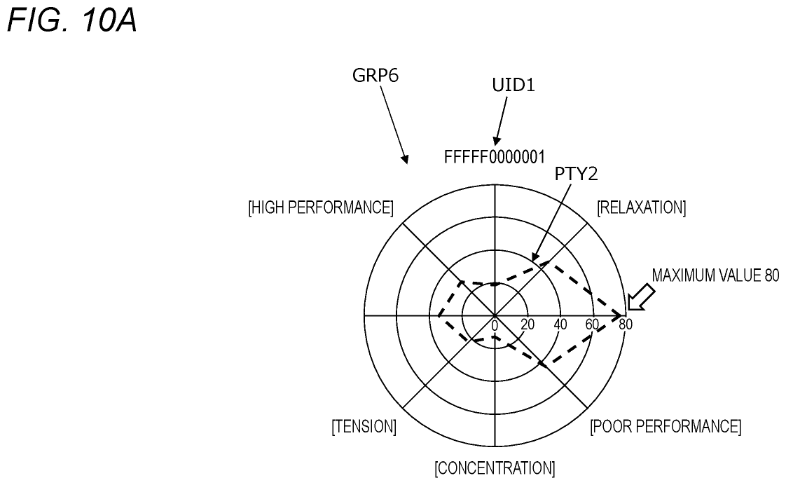

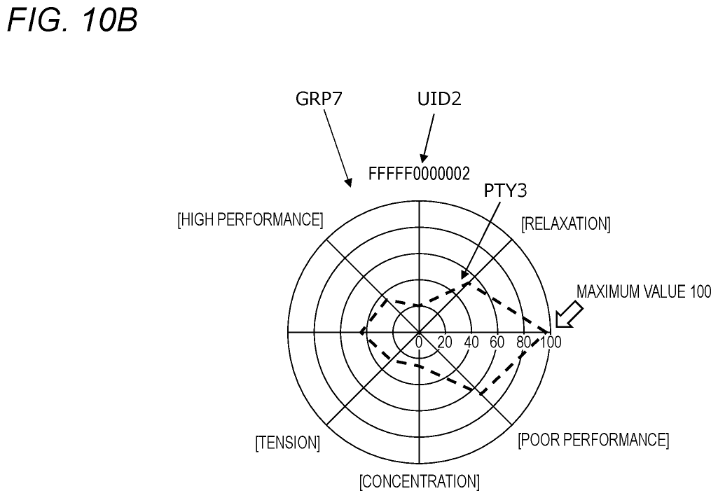

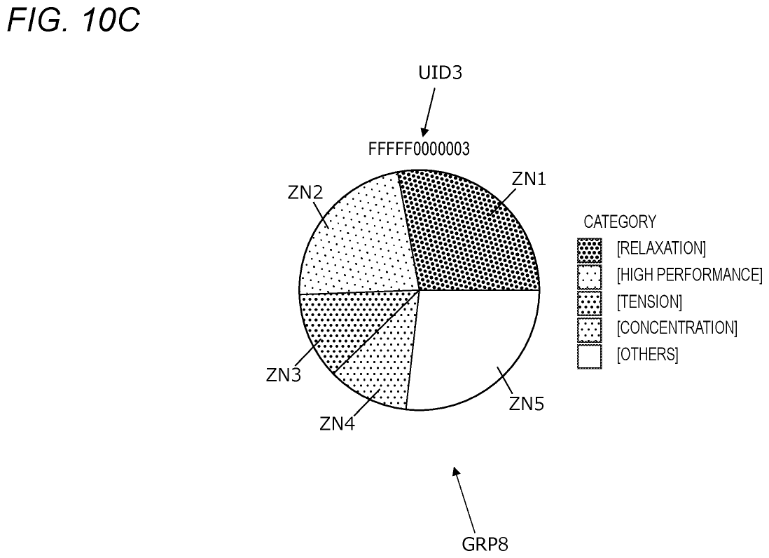

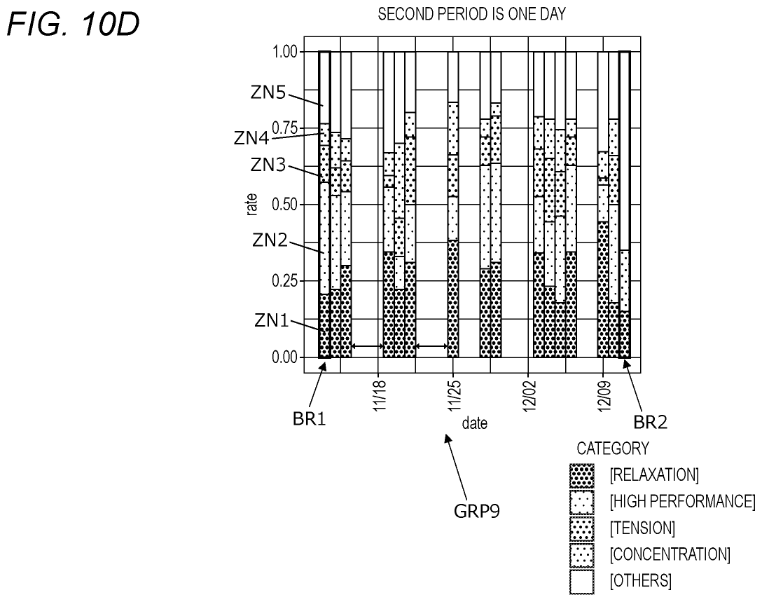

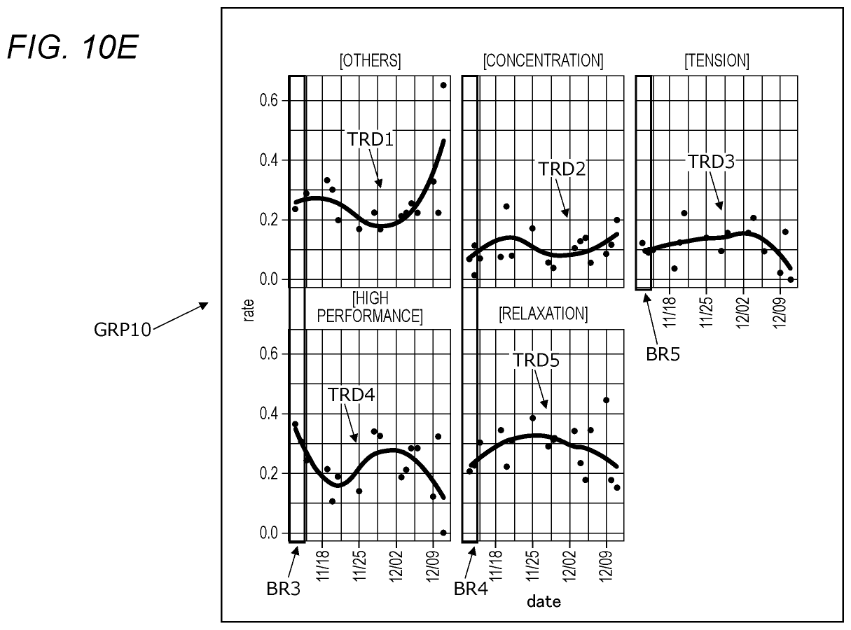

The graph creation unit 4 uses the calculation result of the proportion of each of the plurality of biomarkers of the subject in the second period calculated by the proportion calculation unit 3 to create a biomarker graph (refer to FIGS. 10A, 10B, 10C, 10D, and 10E) for the user to visually grasp the proportion of each of the plurality of biomarkers of the subject at least in the second period, and transmits the graph to the display control unit 5. A specific example of the biomarker graph will be described later in detail with reference to FIGS. 10A to 10E.

The display control unit 5 displays the data of the biomarker graph, which is created by the graph creation unit 4, on the display DP1 via the communication IF circuit 6.

The communication IF circuit 6 constitutes an interface circuit that controls communication with other devices (for example, the biosensors S1, S2, . . . , and the display DP1) that communicate with the biomarker computing device 1. When receiving the data of the biomarker graph transmitted from the display control unit 5, the communication IF circuit 6 transmits the data to the display DP1. In addition, the communication IF circuit 6 receives the sensed biometric data SD1 from the biosensors S1, S2, . . . , and transmits the sensed biometric data SD1 to the processor PRC1. Although not shown in FIG. 1, the sensed biometric data SD1 from the biosensors S1, S2, is input into the biomarker computing device 1 via the communication IF circuit 6.

The memory M1 includes a random access memory (RAM) and a read only memory (ROM), and temporarily stores a program necessary for operating the biomarker computing device 1, and further, data or information generated during the operation. The RAM is, for example, a work memory used during the operation of the processor PRC1. The ROM stores in advance, for example, a program and data for controlling the processor PRC1.

The display DP1 as an example of a display device is a device capable of displaying a display screen of the biomarker graph created by the biomarker computing device 1, and may be, for example, a liquid crystal display (LCD), an organic electroluminescence (EL) device, or another display device.

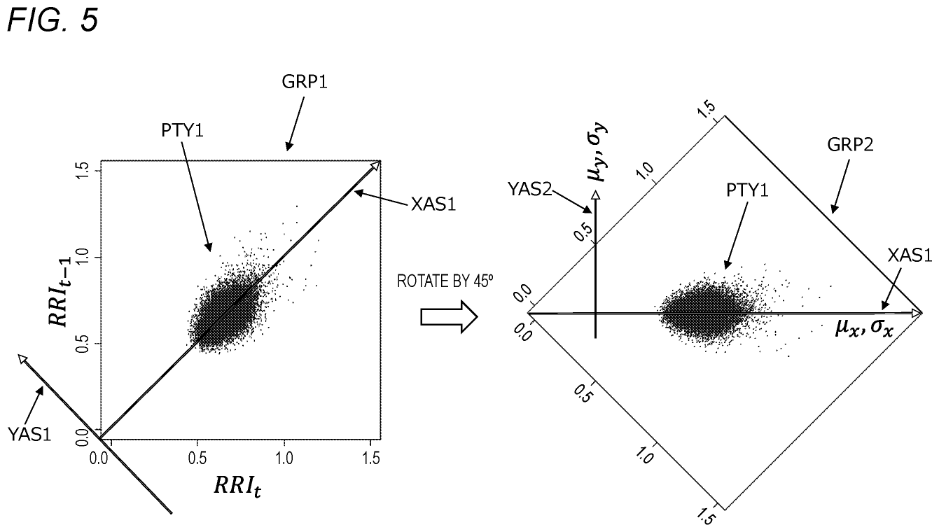

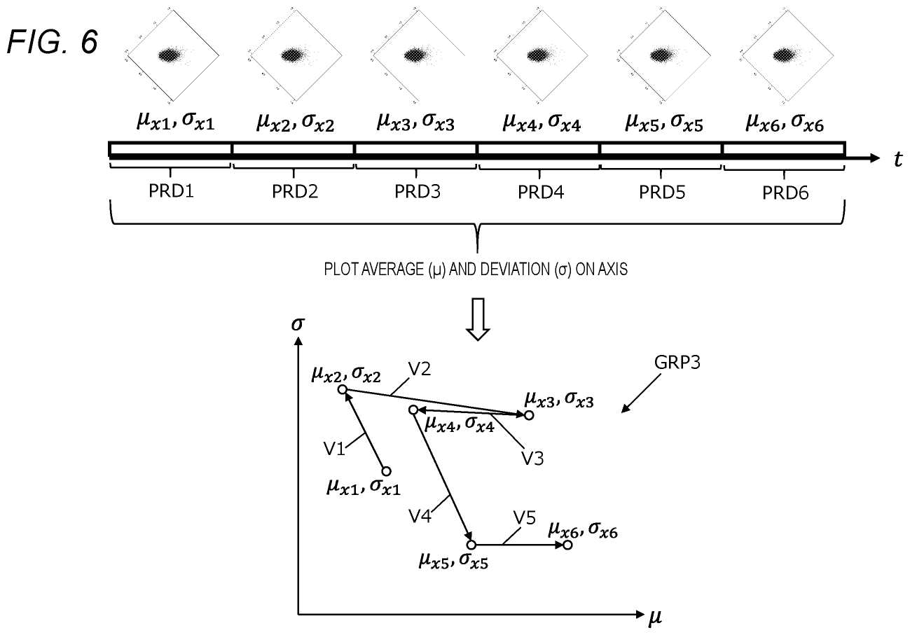

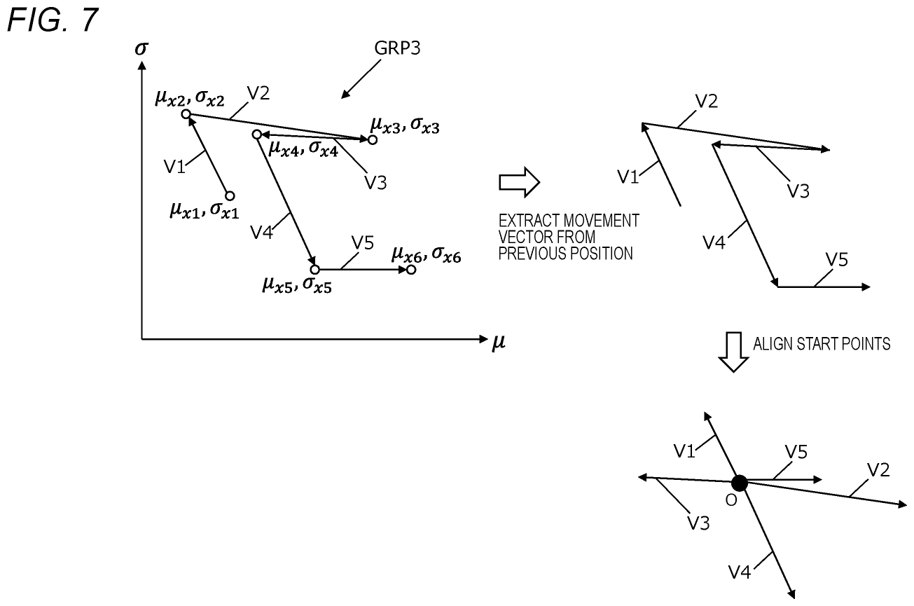

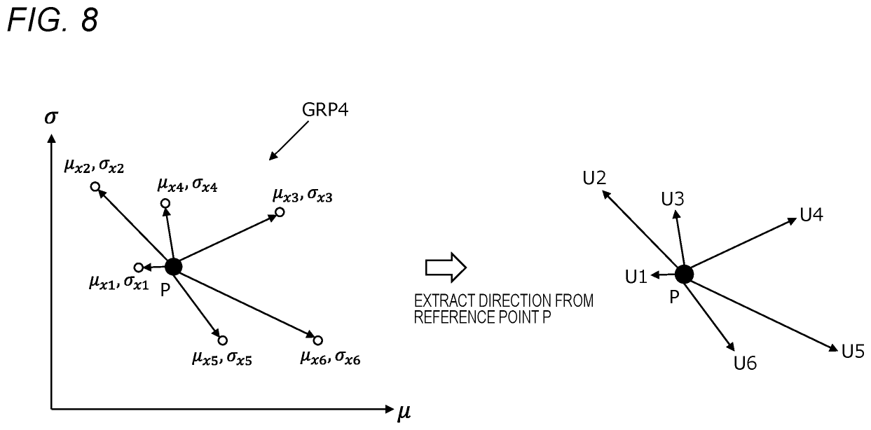

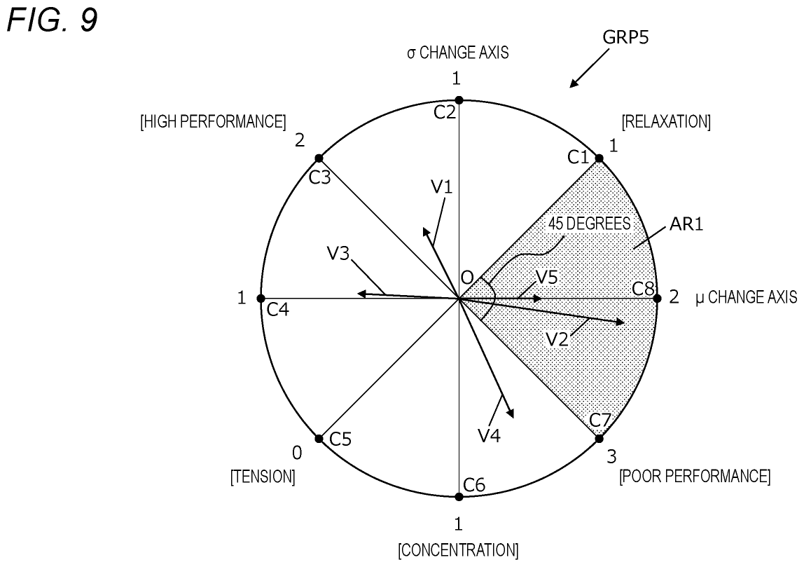

Next, an estimation method of the biomarker of the subject performed by the first marker estimation unit 11 will be described in detail with reference to FIGS. 4 to 9. The estimation method to be described below may be similarly performed by each of the second marker estimation unit 12 to the N-th marker estimation unit 1N different from the first marker estimation unit 11 which receives the sensed biometric data SD1. FIG. 4 is a diagram showing an example of a Lorenz plot of the RRI data. FIG. 5 is a diagram showing a calculation example of an average and a deviation based on the Lorenz plot of the RRI data. FIG. 6 is a diagram showing an example of a time-series plot of a pair including an average and a deviation calculated for each second period. FIG. 7 is a diagram showing a first calculation example of obtaining a change direction (movement vector) for each first period based on the time-series plot of FIG. 6. FIG. 8 is a diagram showing a second calculation example of obtaining the change direction (movement vector) for each first period based on the time-series plot of FIG. 6. FIG. 9 is a diagram showing an example of an eight-classification map in which the movement vectors in FIG. 7 or 8 are aligned with a center point O.

In the following description of FIGS. 4 to 9, the second period is, for example, six times the first period (that is, includes six first periods). That is, when the first period continues six times, the second period is obtained. Six times is merely an example, and it is needless to say that the number of times is not limited to 6 times.

As shown in FIG. 4, the first marker estimation unit 11 receives the sensed biometric data SD1 collected in a first period (for example, one hour). Here, as shown in FIG. 4, the sensed biometric data SD1 input to the first marker estimation unit 11 constitutes a data set in which, for example, a record RC1 including a date time, “RRI” (for example, current RRI data), and “Next RRI” (for example, RRI data after a next sampling interval (that is, a time corresponding to one beat of the heart) has elapsed) is accumulated for a first period. The “Next RRI” may be RRI data after elapse of a next sampling interval or RRI data at a previous sampling interval based on the current RRI data. The first marker estimation unit 11 plots “RRI” and “Next RRI” constituting each record into a two-dimensional graph GRP0 using the sensed biometric data SD1 collected in a first period. In this way, plotting the RRI and Next RRI when one sampling interval changes in time series on a two-dimensional graph is called a Lorenz plot.

In the graph GRP0 of FIG. 4, a horizontal axis represents the “RRI” and a vertical axis represents the “Next RRI”. Specifically, a point A1 having coordinates of (“RRI”, “Next RRI”)=(0.5748517, 0.6106460) of the record RC1 is Lorenz-plotted on the graph GRP0. Similarly, for each record of the sensed biometric data SD1 in a first period, each values of (“RRI”, “Next RRI”) are repeatedly Lorenz-plotted in a position with corresponding coordinates on the graph GRP0, and this process is executed by the first marker estimation unit 11.

A graph GRP1 of FIG. 5 shows results of Lorenz plots of “RRI” and “Next RRI” coordinates corresponding to all of the records constituting the sensed biometric data SD1 in a first period. Based on the Lorenz plots, not only the variation of the “RRI” and the “Next RRI” but also an average thereof can be seen. In the graph GRP1, a horizontal axis represents “RRIₜ” (that is, RRI data in an elapsed time t from a start time of the first period), and a vertical axis represents “RRI₍ₜ₋₁₎” (that is, RRI data measured in a sampling interval previous that of the RRIₜ). In the graph GRP1, for example, a property PTY1 in which the “RRIₜ” is usually plotted in a range of “0.5 to 1.0” and the “RRI₍ₜ₋₁₎” is usually Lorenz-plotted in a range of “0.5 to 1.0” is shown. In other words, many coordinate points of the “RRI” and the “Next RRI” are plotted near a straight line XAS1 indicating a 45-degree direction of the graph GRP1 of “RRIₜ” and “RRI₍ₜ₋₁₎”.

When the graph GRP1 is rotated, for example, clockwise by 45 degrees, a graph GRP2 in FIG. 5 is obtained. That is, in the graph GRP2 in which the straight line XAS1 is a horizontal axis and a straight line YAX1 perpendicular to the straight line XAS1 is a vertical axis, the horizontal axis shows an average μₓ and a deviation σₓ in an x direction (that is, a direction of the straight line XAS1) of the RRI data in the first period, and the vertical axis shows an average μ_{y} and a deviation σ_{y} in a y direction (that is, a direction of the straight line YAS1) of the RRI data in the first period. Therefore, the first marker estimation unit 11 calculates the average μₓ, the deviation σₓ, the average μ_{y}, and the deviation σ_{y} of the RRI data in the first period shown in the graph GRP2. Similarly, the first marker estimation unit 11 calculates the average μₓ, the deviation σₓ, the average μ_{y}, and the deviation σ_{y} of the RRI data in the second period (for example, six first periods).

FIG. 6 shows the average μₓ and the deviation σₓ of the RRI data in each of a first period PRD1, a second first period PRD2, a third first period PRD3, a fourth first period PRD4, a fifth first period PRD5, and a sixth first period PRD6. The first marker estimation unit 11 plots calculation results of the average μₓ and the deviation σₓ of the RRI data in the second period (that is, six first periods) on a graph GRP3 whose horizontal axis is an average μ and whose vertical axis is a deviation σ. Six coordinate points (specifically, (average μₓ₁, deviation σₓ₁), (average μₓ₂, deviation σₓ₂), (average μₓ₃, deviation σₓ₃), (average μₓ₄, deviation σₓ₄), (average μₓ₅, deviation σₓ₅), (average μₓ₆, deviation σₓ₆)) in the first period PRD1 to the sixth first period PRD1 are plotted on the graph GRP3. For example, when sensing with the biosensors S1, S2, . . . is performed, the subject may move (for example, ahead of the subject moves momentarily), which may disturb a part of the sensed biometric data SD1, and the RRI data for each beat may be disturbed, but stability (reliability) of the data can be obtained and improvement in estimation accuracy is expected when the biomarker is estimated by calculating the average and deviation of RRI data for a first period (for example, one hour) and averaging the RRI data.

As shown in FIGS. 6 and 7, the first marker estimation unit 11 calculates movement vectors V1, V2, V3, V4, and V5 from one coordinate point to another coordinate point in order of being plotted on the graph GRP3. The movement vector V1 is a movement vector from the coordinate point (average μₓ₁, deviation σₓ₁) to the coordinate point (average μₓ₂, deviation σₓ₂). The movement vector V2 is a movement vector from the coordinate point (average μₓ₂, deviation σₓ₂) to the coordinate point (average μₓ₃, deviation σₓ₃). The movement vector V3 is a movement vector from the coordinate point (average μₓ₃, deviation σₓ₃) to the coordinate point (average μₓ₄, deviation σₓ₄). The movement vector V4 is a movement vector from the coordinate point (average μₓ₄, deviation σₓ₄) to the coordinate point (average μₓ₅, deviation σₓ₅). The movement vector V5 is a movement vector from the coordinate point (average μₓ₅, deviation σₓ₅) to the coordinate point (average μₓ₆, deviation σₓ₆).

As an example of extraction and mapping of first movement vectors, the first marker estimation unit 11 extracts the movement vectors V1 to V5 from the graph GRP3, aligns start points of the respective movement vectors V1 to V5, and maps the movement vectors V1 to V5 when the start points thereof are aligned to an eight-classification map GRP5 (refer to FIG. 9). The first marker estimation unit 11 outputs, based on the eight-classification map GRP5, the biomarker estimation results of the subject in the second period.

As an example of extraction and mapping of second movement vectors, the first marker estimation unit 11 may set a predetermined reference point P as shown in a graph GRP4 of FIG. 8, and extract movement vectors U1, U2, U3, U4, U5, and U6 from the reference point P to the coordinate point (average μₓ₁, deviation μₓ₁), the coordinate point (average μₓ₂, deviation σₓ₂), the coordinate point (average μₓ₃, deviation σₓ₃), the coordinate point (average μₓ₄, deviation σₓ₄), the coordinate point (average μₓ₅, deviation σₓ₅), and the coordinate point (average μₓ₆, deviation σₓ₆). The reference point P is, for example, a coordinate point corresponding to an average value of the coordinate point (average μₓ₁, deviation σₓ₂), the coordinate point (average μₓ₂, deviation σₓ₂), the coordinate point (average μₓ₃, deviation σₓ₃), the coordinate point (average μₓ₄, deviation σₓ₄), the coordinate point (average μₓ₅, deviation σₓ₅), and the coordinate point (average μₓ₆, deviation σₓ₆). In addition, as the reference point P, for example, an average and a deviation of the heart rate variability data at rest of the subject may be measured in advance, and a coordinate point specified by the average and the deviation may be set as the reference point P. The reference point P is not limited to the average value of the coordinate points described above or the coordinate point specified by the average and deviation of the heart rate variability data at rest. The first marker estimation unit 11 may map the movement vectors U1 to U6 to the eight-classification map GRP5. An example of mapping the movement vectors U1 to U6 to the eight-classification map is omitted.

The eight-classification map GRP5 of FIG. 9 is a circular map having eight axes (corresponding to diameters) intersecting with one another at the center point O, and classifies and shows the biomarkers indicating the physical and mental states of the subject. In the eight-classification map GRP5, the count value of the appearance frequency of the biomarker is set corresponding to at least a part of intersection points C1, C2, C3, C4, C5, C6, C7, and C8 between the eight axes and the circumference. Specifically, the “relaxation” is set at the intersection point C1, the “high performance” is set at the intersection point C3, the “tension” is set at the intersection point C5, the “concentration” is set at the intersection point C6, and the “poor performance” is set at the intersection point C7.

The first marker estimation unit 11 maps the movement vectors V1 to V5 described with reference to FIG. 7 or 8 (for example, refer to FIG. 7) on the eight-classification map GRP5 such that the start points of the movement vectors V1 to V5 coincide with the center point O. The first marker estimation unit 11 reads out a count value corresponding to an axis (for example, an axis in a direction from the center point O toward the intersection point C8 in FIG. 9) in a center direction of a fan-shaped portion AR1 whose interior angle around the center point O is 90 degrees while moving the fan-shaped portion AR1 clockwise or counterclockwise by 45 degrees. For example, when the fan-shaped portion AR1 is specified by the intersection point C1, the center point O, and the intersection point C7 (refer to FIG. 9), the first marker estimation unit 11 reads the number of the movement vectors V2 and V5 (here, a value of 2) mapped in the fan-shaped portion AR1 as a count value corresponding to the intersection point C8. When the fan-shaped portion AR1 is specified by the intersection point C8, the center point O, and the intersection point C6, the first marker estimation unit 11 reads the number of the movement vectors V5, V2, and V4 (here, a value of 3) mapped in the fan-shaped portion AR1 as a count value corresponding to the intersection point C7. Thereafter, similarly, the first marker estimation unit 11 reads the number of the movement vector V5 (here, a value of 1) mapped in the fan-shaped portion AR1 as a count value corresponding to the intersection point C1.

The first marker estimation unit 11 refers to the settings of the “relaxation” at the intersection point C1, the “high performance” at the intersection point C3, the “tension” at the intersection point C5, the “concentration” at the intersection point C6, and the “poor performance” at the intersection point C7, to estimate the appearance frequency of “relaxation” as “1”, the appearance frequency of “high performance” as “2”, the appearance frequency of “tension” as “0”, the appearance frequency of “concentration” as “1”, and the appearance frequency of “poor performance” as “3” within the second period.

Next, types of the graphs created by the graph creation unit 4 of the biomarker computing device 1 according to the first embodiment will be described with reference to FIGS. 10A to 10E. FIGS. 10A and 10B are diagrams of radar charts each showing an example of appearance frequencies of a plurality of biomarkers of a subject in a unit period. FIG. 10C is a diagram of a pie chart showing an example of appearance frequencies of biomarkers of a subject in a unit period. FIG. 10D is a diagram of a bar graph showing an example of appearance frequencies of biomarkers of a subject in a unit period. FIG. 10E is a diagram of a line graph showing an example of appearance frequencies of biomarkers of a subject in a unit period. In the description of FIGS. 10A to 10E, the same elements are denoted by the same reference numerals, and the description thereof will be simplified or omitted.

FIG. 10A shows a radar chart GRP6 with a property PTY2 of a rate calculated based on an appearance frequency of each of a plurality of biomarkers of a subject whose identification number UID1 is “FFFFF0000001” in a second period (for example, one day). A circle shown in the radar chart GRP6 corresponds to the eight-classification map GRP5 of FIG. 9. In the radar chart GRP6, a maximum value among the count values corresponding to the eight axes described with reference to FIG. 9 is 80, and the count values corresponding to the eight axes are plotted and connected by broken lines. Therefore, according to the radar chart GRP6, it is possible to visually show a rate of appearance of each biomarker of the subject whose identification number UID1 is “FFFFF0000001” in the second period (for example, one day) with respect to a count value with the maximum value 80 (that is, at what frequency the corresponding biomarker is seen in the second period).

FIG. 10B shows a radar chart GRP7 with a property PTY3 of a rate calculated based on an appearance frequency of each of a plurality of biomarkers of a subject whose identification number UID2 is “FFFFF0000002” in a second period (for example, one day). A circle shown in the radar chart GRP7 corresponds to the eight-classification map GRP5 of FIG. 9. In the radar chart GRP7, a maximum value among the count values corresponding to the eight axes described with reference to FIG. 9 is 100, and the count values corresponding to the eight axes are plotted and connected by broken lines. Therefore, according to the radar chart GRP7, it is possible to visually show a rate of appearance of each biomarker of the subject whose identification number UID2 is “FFFFF0000002” in the second period (for example, one day) with respect to a count value with the maximum value 100 (that is, at what frequency the corresponding biomarker is seen in the second period). In addition, as compared with FIG. 10A, even in a case where the maximum value of the appearance frequency of the biomarker is different for being 80 and 100 (in other words, even in a case where the number of the first periods in which the estimation of the biomarker is possible is different for each subject), the appearance frequency of each of the biomarkers is indicated by a rate, so that, for example, biomarkers of a plurality of persons in a certain period such as the second period can be easily compared. The reason why the first period in which the estimation of the biomarker is possible is different for each subject is that, for example, the number of times the sensed biometric data SD1 is acquired by the biosensors S1, S2, . . . , is different between such as a person who basically performs desk work during one day and a person who frequently leaves a seat due to a conference, a business trip, or the like.

FIG. 10C shows a pie chart GRP8 of rates calculated based on appearance frequencies of a plurality of biomarkers of a subject whose identification number UID3 is “FFFFF000003” in a second period (for example, one day). In the pie chart GRP8, an area ZN1 of the “relaxation”, an area ZN2 of the “high performance”, an area ZN3 of the “tension”, an area ZN4 of the “concentration”, and an area ZN5 of “others” are displayed in different colors according to the rates. Therefore, according to the pie chart GRP8, the rate of each biomarker of the subject whose identification number UID3 is “FFFFF000003” in the second period (for example, one day) can be presented visually and easily.

FIG. 10D shows, as a bar graph, a bar graph GRP9 in which rates calculated based on appearance frequencies of a plurality of biomarkers of a certain subject for each second period (for example, one day) are collected for a unit period (for example, a third period longer than the second period). In FIG. 10D, the third period is, for example, one month, but is not limited to one month as long as the third period is longer than the second period. Specifically, the bar graph GRP9 is configured such that bar graphs created for each second period from a bar graph BR1 corresponding to a first second period in the third period to a bar graph BR2 corresponding to a last second period in the third period are arranged. Therefore, according to the bar graph GRP9, a transition of each biomarker of the subject for each second period (for example, daily) throughout the entire third period can be visually and easily presented.

FIG. 10E shows a line graph GRP10 in which line graphs TRD1, TRD2, TRD3, TRD4, and TRD5 are integrated, and each of the line graphs TRD1, TRD2, TRD3, TRD4, and TRD5 is configured by collecting, for each of a plurality of biomarkers of a certain subject in each second period (for example, one day), a rate of the biomarker in the second period for a unit period (for example, a third period longer than the second period). In FIG. 10E, the third period is, for example, one month, but is not limited to one month as long as the third period is longer than the second period. In FIG. 10E, the biomarkers are, for example, “others”, “concentration”, “tension”, “high performance”, and “relaxation”, and the line graphs for the five biomarkers are shown individually for TRD1, TRD2, TRD3, TRD4, and TRD5. For the line graphs TRD1, TRD2, TRD3, TRD4, and TRD5 constituting the line graph GRP10, a start timing and an end timing of the third period are the same (that is, made common). Specifically, the line graphs TRD1 and TRD4 are configured such that bar graphs created for each second period from a bar graph BR3 corresponding to a first second period in the third period to a bar graph corresponding to a last second period in the third period are arranged. Similarly, the line graphs TRD2 and TRD5 are configured such that bar graphs created for each second period from a bar graph BR4 corresponding to the first second period in the third period to a bar graph corresponding to the last second period in the third period are arranged. The line graph TRD3 is configured such that bar graphs created for each second period from a bar graph BR5 corresponding to the first second period in the third period to a bar graph corresponding to the last second period in the third period are arranged. Therefore, according to the line graph GRP10, not only a transition of each biomarker of the subject in each second period (for example, daily) can be presented visually and easily over the entire third period, but also a trend of the transition of the biomarker (in other words, emotion) in each second period (that is, daily) can be presented in an easily understandable manner.

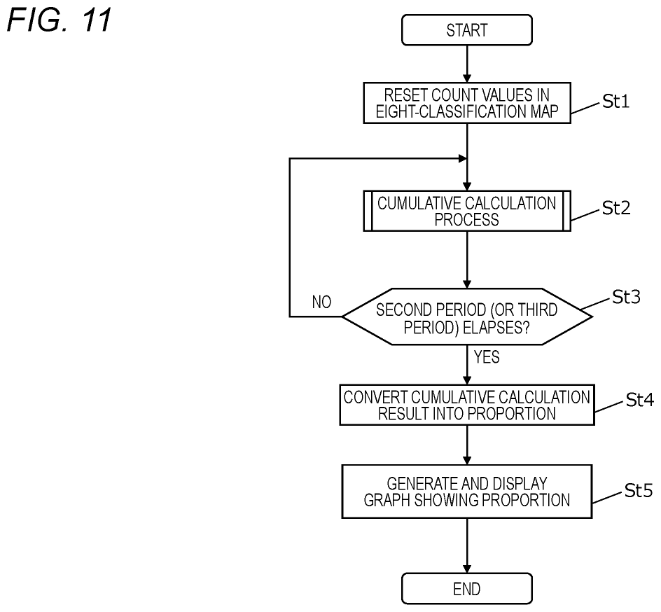

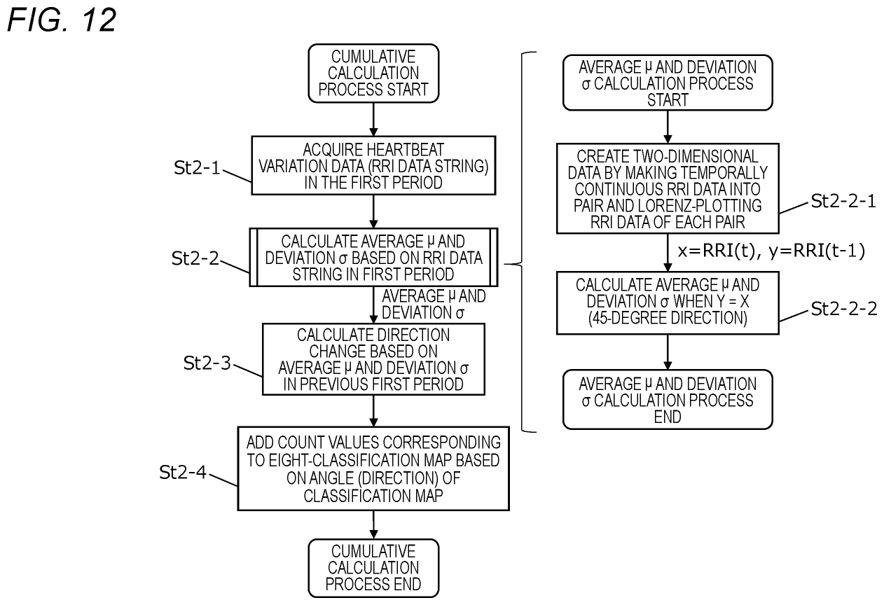

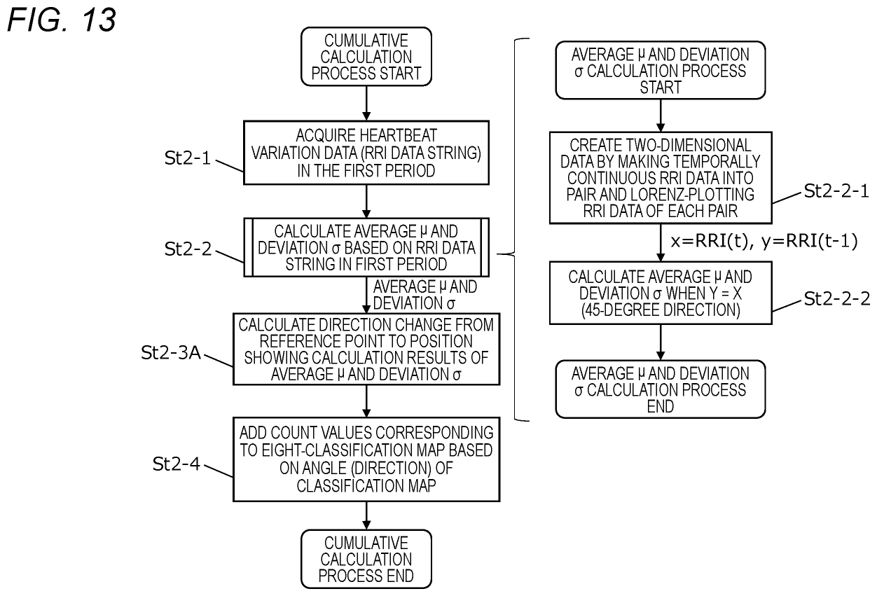

Next, an operation procedure of the biomarker computing device 1 according to the first embodiment will be described with reference to FIGS. 11 to 13. FIG. 11 is a flowchart showing an example of an overall operation procedure of the biomarker computing device 1 according to the first embodiment. FIG. 12 is a flowchart showing a first example of a cumulative calculation process of FIG. 11. FIG. 13 is a flowchart showing a second example of the cumulative calculation process of FIG. 11. In the description of FIGS. 12 and 13, the same processes are denoted by the same step numbers, the description thereof will be simplified or omitted, and different contents will be described. The processes of FIGS. 12 and 13 are mainly performed by the first marker estimation unit 11, and may be performed similarly by each of the second marker estimation unit 12 to the N-th marker estimation unit 1N.

In FIG. 11, the biomarker computing device 1 resets (initializes) the count values corresponding to the eight axes of the eight-classification map GRP5 described with reference to FIG. 9 (SU). After step St1, the biomarker computing device 1 performs a cumulative calculation process for each first period using the sensed biometric data SD1 received within the first period (St2). The biomarker computing device 1 determines whether the cumulative calculation process for each first period is performed for the second period (St3). For example, when the first period is one hour and the second period is one day, it is determined whether the cumulative calculation process is performed 24 times in total. Even if the second period is one day, when working hours of the subject in a company are nine hours in total from 9 a.m. to 6 p.m., the second period may be substantially regarded as nine hours.

When the cumulative calculation process for each first period is not executed for the second period (NO in St3), processing of step St2 is repeated until the cumulative calculation process for each first period is executed for the second period.

On the other hand, when it is determined that the cumulative calculation process for each first period is executed for the second period (YES in St3), the biomarker computing device 1 counts the appearance frequency of each of the plurality of biomarkers in the second period, and calculates the proportion of each biomarker (that is, the physical and mental state) based on the count value (St4). The biomarker computing device 1 uses the calculation result of the proportion of each of the plurality of biomarkers of the subject in the second period to create a biomarker graph for the user to visually grasp at least the proportion of each of the plurality of biomarkers of the subject in the second period, and displays the biomarker graph on the display DP1 (St5).

In FIG. 12, the biomarker computing device 1 acquires RRI data indicating the variation of the heartbeat sounds in the first period (St2-1). The biomarker computing device 1 calculates the average μ and the deviation σ of the RRI data in a first period acquired in step St2-1 (St2-2, refer to FIG. 5).

Here, step St2-2 will be described in detail.

The biomarker computing device 1 creates two-dimensional data of the RRI data in a first period acquired in step St2-1 by making two pieces of temporally continuous RRI data among the RRI data in a first period into a pair and Lorenz-plotting the RRI data of each pair (refer to FIG. 4) (St2-2-1, refer to FIG. 5). In FIG. 5, the horizontal axis (x-axis) shows RRI(t) indicating RRI data at a time point t, and the vertical axis (y-axis) shows RRI(t−1) indicating RRI data at a time point (t−1).

The biomarker computing device 1 calculates an average μ and a deviation σ when y=x (that is, in the 45-degree direction) in the two-dimensional data created in step St2-2-1 (St2-2-2). This is because, as described above, many coordinates of (RRI(t), RRI(t−1)) are distributed near a straight line of a linear function which is y=x.

The biomarker computing device 1 maps, to two-dimensional data, a coordinate point determined by values of (average deviation 6) for each first period calculated in steps St2-2. The biomarker computing device 1 calculates a movement vector from a coordinate point determined by values of (average deviation 6) in a previous first period to a coordinate point determined by values of (average deviation 6) in a current first period over the first periods constituting the second period (St2-3, refer to FIG. 7).

The biomarker computing device 1 maps the plurality of movement vectors calculated in steps St2-3 to the eight-classification map GRP5 (refer to FIG. 9). The biomarker computing device 1 reads out the count value corresponding to the axis in the center direction of the fan-shaped portion AR1 whose interior angle around the center point O of the eight classification map GRP5 is 90 degrees while moving the fan-shaped portion AR1 clockwise or counterclockwise by 45 degrees. The biomarker computing device 1 counts and outputs the biomarker estimation results of the subject in the second period based on the eight-classification map GRP5 after mapping (St2-4).

In FIG. 13, the biomarker computing device 1 sets the predetermined reference point P, and calculates the movement vectors U1, U2, U3, U4, U5, and U6 from the reference point P to respective coordinate points each of which is determined by the values of (average deviation σ) in each first period calculated in step St2-2 (St2-3A, refer to FIG. 8). Since the subsequent processing is the same as that in FIG. 12, the description thereof will be omitted.

As described above, the biomarker computing device 1 according to the first embodiment receives biometric data (for example, sensed biometric data SD1) of a subject measured by the biosensors S1, S2, . . . . The biomarker computing device 1 uses the received biometric data in a first period (for example, one hour) to estimate a biomarker indicating a physical and mental state of the subject. The biomarker computing device 1 collects, in a second period (for example, one day) longer than the first period, a plurality of biomarker estimation results estimated for respective first periods, and calculates, based on appearance frequencies of the physical and mental states indicated respectively by the plurality of biomarker estimation results, a proportion of each of the physical and mental states. The biomarker computing device 1 creates a graph indicating the proportions of the physical and mental states, and displays the created graph on the display DP1.

Accordingly, since the biomarker computing device 1 can show the biomarker of the subject in a unit period (for example, the second period) by a rate, a summary result of the biomarker in the unit period such as a transition of the biomarker can be output, and thus an analysis of the biomarker of the subject can be efficiently supported. For example, as compared with a case where a result of the biomarker in the first period is simply accumulated and displayed in a graph, the biomarker computing device 1 can visually show the rate of the biomarker of the subject while reducing variation in the biomarker of the subject in a second period longer than the first period. Therefore, it is possible to intuitively and visually show how a change of the biomarker of a certain subject in one day of a certain day is balanced, or a transition about how the biomarker of a certain subject changes throughout one month.

The biometric data includes a plurality of types of biometric data. The biomarker computing device 1 includes a plurality of biomarker estimation units in which combinations of the biometric data used for estimation of the biomarker among the plurality of types of biometric data are different. Accordingly, the biomarker computing device 1 can estimate the biomarker of the subject in various ways and with high accuracy by changing the type of the sensed biometric data SD1 used for the estimation of the biomarker.

The biometric data is data indicating heart rate variability of the subject measured by the biosensors S1, S2, for each predetermined time. Accordingly, since the biomarker computing device 1 can use the RRI data of the subject, the biomarkers (that is, emotions) of the subject in the second period can be estimated with high accuracy.

The physical and mental state includes at least two among relaxation, concentration, and tension. Accordingly, the biomarker computing device 1 can visually show rates of appearance frequencies of at least two among the relaxation, the concentration, and the tension, which are easily measured as physical and mental states of a subject in a habitual manner.

The biomarker computing device 1 outputs the biomarker estimation result in the first period in a form of binary data. Therefore, the biomarker computing device 1 can efficiently calculate the proportions of the biomarkers of the subject since the biomarker estimation results are standardized into binary data.

In addition, the biomarker computing device 1 normalizes the biomarker estimation result in each first period estimated by each of the plurality of biomarker estimation units (the first marker estimation unit 11 to the N-th marker estimation unit 1N) and collects the estimation results for the second period. Therefore, the biomarker computing device 1 can standardize ranges of values of the biomarker estimation results regardless of a type of the biomarker estimation method of each of the first marker estimation unit 11 to the N-th marker estimation unit 1N, and thus can calculate the biomarker estimation result of the subject with high accuracy.

In addition, the biomarker computing device 1 creates a graph in which the proportion calculation results of the physical and mental states in each second period are arranged for a third period (for example, one month) longer than the second period. Accordingly, the biomarker computing device 1 can visually and easily present a transition of each biomarker of the subject in each second period (for example, daily) throughout an entire third period.

In addition, the biomarker computing device 1 estimates the biomarker based on a Lorenz plot of a first statistical parameter (for example, an average μ and a deviation σ) calculated based on the biometric data in the previous first period and a second statistical parameter (for example, an average μ and a deviation σ) calculated based on the biometric data in the first period to be estimated. Accordingly, the biomarker computing device 1 can calculate the biomarker of the subject with high accuracy while reducing variations in the received sensed biometric data SD1 of the subject.

When the biomarker cannot be estimated, the biomarker computing device 1 outputs a value of 0 as the biomarker. Accordingly, the biomarker computing device 1 can appropriately calculate the biomarker of the subject using only estimation-enabled values by excluding an influence of the second period in which the biomarker cannot be estimated.

Although the embodiments have been described with reference to the accompanying drawings, the present disclosure is not limited thereto. It is apparent to those skilled in the art that various modifications, corrections, substitutions, additions, deletions, and equivalents can be conceived within the scope described in the claims, and it is understood that such modifications, substitutions, additions, deletions, and equivalents also fall within the technical scope of the present disclosure. Further, components in the above-described embodiment may be optionally combined within a range not departing from the spirit of the invention.

The present application is based on a Japanese patent application filed on Feb. 25, 2020 (Japanese Patent Application No. 2020-029882), the contents of which are incorporated by reference in the present application.

### INDUSTRIAL APPLICABILITY

The present disclosure is useful as a biomarker computing device and a biomarker computing method for outputting a summary result of a biomarker of a subject in a unit period and efficiently supporting an analysis of the biomarker of the subject.

### REFERENCE SIGNS LIST

1: biomarker computing device
2: cumulative monitoring unit
3: proportion calculation unit
4: graph creation unit
5: display control unit
6: communication IF circuit
11: first marker estimation unit
12: second marker estimation unit
1N: N-th marker estimation unit
100: biomarker monitoring system
DP1: display
M1: memory
PRC1: processor
S1, S2: biosensor
SD1: sensed biometric data

1. A biomarker computing device comprising:
an input unit configured to input biometric data of a subject measured by a biosensor;
a biomarker estimation unit configured to estimate, by using the input biometric data in a first period, a biomarker indicating a physical and mental state of the subject;
a computation unit configured to collect, in a second period longer than the first period, a plurality of biomarker estimation results estimated in each first period, and calculate, based on appearance frequencies of the physical and mental states indicated respectively by the plurality of biomarker estimation results, a proportion of each of the physical and mental states;
a graph creation unit configured to create a graph showing the proportion of each of the physical and mental states; and
a display control unit configured to display the created graph on a display device.

2. The biomarker computing device according to claim 1, wherein
the biometric data includes a plurality of types of biometric data, and
the biomarker estimation unit includes a plurality of biomarker estimation units in which combinations of the biometric data used for estimation of the biomarker among the plurality of types of biometric data are different.

3. The biomarker computing device according to claim 1, wherein
the biometric data is data indicating heart rate variability of the subject measured by the biosensor for each predetermined time.

4. The biomarker computing device according to claim 1, wherein
the physical and mental states include at least two among relaxation, concentration, and tension.

5. The biomarker computing device according to claim 1, wherein
the biomarker estimation unit outputs the biomarker estimation result in the first period in a form of binary data.

6. The biomarker computing device according to claim 2, wherein
the computation unit normalizes the biomarker estimation result in each of the first periods estimated by each of the plurality of biomarker estimation units and collects the normalized estimation result for the second period.

7. The biomarker computing device according to claim 1, wherein
the graph creation unit creates a graph in which the proportion calculation results of the physical and mental states in each of the second periods are arranged for a third period longer than the second period.

8. The biomarker computing device according to claim 1, wherein
the biomarker estimation unit estimates the biomarker based on a Lorenz plot of a first statistical parameter calculated based on the biometric data in the previous first period and a second statistical parameter calculated based on the biometric data in the first period to be estimated.

9. The biomarker computing device according to claim 5, wherein
the biomarker estimation unit outputs a value of 0 as the biomarker in a case that estimation of the biomarker is impossible.

10. A biomarker computing method to be executed by a biomarker computing device, the biomarker computing method comprising:
inputting biometric data of a subject measured by a biosensor;
estimating, by using the input biometric data in a first period, a biomarker indicating a physical and mental state of the subject;
collecting, in a second period longer than the first period, a plurality of biomarker estimation results estimated in each first period, and calculating, based on appearance frequencies of the physical and mental states indicated respectively by the plurality of biomarker estimation results, a proportion of each of the physical and mental states;
creating a graph showing the proportion of each of the physical and mental states; and
displaying the created graph on a display device.
